(19) United States
(12) Patent Application Publication
(10) Pub. No.: US 2018/0228897 A1
(43) Pub. Date: Aug. 16, 2018

(54) Compositions for Vapor Phase Delivery of Active Compounds, Methods of Making and Using Same, and Kits Comprising Same

(71) Applicant: Atmovita, LLC, Blue Point, NY (US)

(72) Inventors: Stephen Alan Boyd, Manhasset, NY (US); Yevgenity Ovstrovskiy, Bringhton Beach, NY (US)

(21) Appl. No.: 15/750,377

(22) PCT Filed: Aug. 3, 2016
(86) PCT No.: PCT/US2016/045344

§ 371 Date: Feb. 5, 2018

Related U.S. Application Data
(60) Provisional application No. 62/200,471, filed on Aug. 3, 2015.

(51) Int. Cl. A61K 47/10; A61K 31/4188; A61K 31/519; A61K 31/455; A61K 31/05; A61K 31/522; A61K 31/192; A61K 31/60; A61K 31/51; A61K 31/197; A61K 31/4415; A61K 31/4015; A61K 31/593; A61K 31/198; A61K 31/525; A61K 38/05; A61K 9/00

(52) U.S. Cl. CPC A61K 47/10; A61K 9/007; A61K 31/519; A61K 31/455; A61K 31/05; A61K 31/522; A61K 31/192; A61K 31/60; A61K 31/51; A61K 31/197; A61K 31/4415; A61K 31/4015; A61K 31/593; A61K 31/198; A61K 31/525; A61K 38/05; A61K 31/4188

(57) ABSTRACT

Compositions for vapor phase delivery of one or more active compounds. The compositions comprise one or more polyol and one or more active compound. Optionally, the compounds further comprise one or more disruptive compound. The compositions can be used in methods of vapor phase delivery of one or more active compound to an individual. Also provided are kits comprising one or more of the compositions and instructions for use of the composition(s).

### CROSS REFERENCE TO RELATED APPLICATIONS

This application claims priority to U.S. Provisional Application No. 62/200,471, filed on Aug. 3, 2015, the disclosure of which is hereby incorporated by reference.

### FIELD OF THE DISCLOSURE

The disclosure generally relates to vaping compositions. More particularly the disclosure generally relates to polyol vaping compositions.

### BACKGROUND OF THE DISCLOSURE

Vaporization is a term that has come into common parlance of late. It refers to the phase change associated with achieving the (often azeotropically driven) mean mixture boiling point of a number of fluids. This method has recently been popularized due to the ability of the solvents to act as a carrier medium in the vapor phase of nicotine. This property can extend to many other molecules that are soluble in polar solvents such as water, 1,2,3-propanetriol, ethanol and the like.

Given the nature of the transition from the liquid phase to the vapor phase, a concomitant reduction in active-compound concentration is suffered, due to the density of the vapor phase for polar or supported polar compounds. The drop in density is roughly 1/1000 of that of the liquid phase for these compounds.

Conventional formulations (e.g., 70% glycerin, 30% propylene glycol (v/v)) are inadequate to support anything other than highly compatible compounds, i.e. compounds that are polar and thus miscible in glycerin and/or propylene glycol. Further, oil soluble compounds or more complex molecules are completely incompatible with the current formulations.

### SUMMARY OF THE DISCLOSURE

The present disclosure provides a composition for vapor phase delivery of active compounds comprising one or more polyol, optionally, one or more disruptive compound; and one or more active compound. A method for vapor phase delivery of active compounds is also provided. It is advantageous to take measures to increase the solubility of an active compound or active compounds within a composition that includes components that serve as excipients such that true solubilization of the compound(s) occurs and the activity of the active compounds are retained upon transition to the vapor phase. The present disclosure is based on the result that the solubility of active compounds can be increased by surprising and unexpected amounts in compositions of the present disclosure.

In an aspect, the present disclosure provides compositions for vapor phase delivery of active compounds. The compositions comprise one or more polyol and one or more active compound. Optionally, the compositions comprise one or more disruptive compound. Optionally, the compositions comprise one or more additional component.

In an aspect, the present disclosure provides methods for making the compositions. The compositions can be made by mixing together the individual components (e.g., polyol(s), optionally, disruptive compounds(s), active compound(s), optionally, water, and any other optional components). The individual components can be combined in any order. The compositions may be heated at any point during the mixing of the components or after the components are mixed together.

In an aspect, the present disclosure provides methods of using the compositions. For example, the compositions are used in methods of administering active compound(s) in the vapor phase to an individual. The administration to the individual is via the transalveolar tissue passage of the individual and/or the mucosal lining of the airway of the individual. The administration may result in at least a detectible amount of the active compound(s) and/or its/their derivative(s) (e.g., metabolite) in the blood stream (e.g., serum and/or blood plasma) and/or urine and/or central nervous system (CNS) fluid of the individual. In an embodiment, the active compound(s) is/are not administered to the individual as an aerosol.

In an aspect, the present disclosure provides kits. A kit can comprise one or more compound of the present disclosure and instructions for its use. Optionally, a kit includes a vaporizing apparatus (e.g., an electronic vaporizing apparatus).

### BRIEF DESCRIPTION OF THE FIGURES

For a fuller understanding of the nature and objects of the disclosure, reference should be made to the following detailed description taken in conjunction with the accompanying FIGURE.

FIG. 1. Serum concentration of an active compound (vitamin B12) in an individual (determined by ELISA) as function of time after vaping a composition of the instant disclosure comprising vitamin B12.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The present disclosure provides compositions for vapor phase delivery of active compounds. Also provided are methods for making and using the compositions and kits comprising the compositions.

It is advantageous to take measures to increase the solubility of an active compound or active compounds within a composition that includes components that serve as excipients such that true solubilization of the compound(s) occurs and the activity of the active compounds are retained upon transition to the vapor phase. The present disclosure is based on the result that the solubility of active compounds can be increased by surprising and unexpected amounts. For example, we achieved a surprising and unexpected 600% increase in solubility for cyanocobalamin crystal (B12), as much as a 15,000% increase in solubility for folic acid (B9), and comparable increases for D3 (22,000%, which is oil-soluble only, but soluble with the use of ß-propoxy cyclodextrin) and B7 in the present compositions.

In an aspect, the present disclosure provides compositions for vapor phase delivery of active compounds. The compositions comprise one or more polyol and one or more active compound. Optionally, the compositions comprise one or more disruptive compound. Optionally, the compositions comprise one or more additional component.

In an embodiment, the composition comprises a polyol, a disruptive compound, and an active compound. Optionally, the composition further comprises one or more additional component.

A variety of polyols can be used in the compositions. Without intending to be bound for any particular theory, it is considered that support arises at least in part from the hydrogen bonding that occurs between the multiple hydroxyl groups of the polyol and other functional groups, if present, and the accompanying solute. The polyols are hydrocarbon compounds. The polyols have, for example, 2 to 20 hydroxyl groups, including all integer number of hydroxyl groups therebetween. The polyols have, for example, a linear, 3 to 6 carbon backbone. This is desirable because carbon backbones of linear polyols greater than C₆ have been found to increase viscosity despite the addition of a disruptive compound such as ethanol. The polyols may have other functional groups. For example, the polyol has one or more complementary polar moiety functional groups (e.g., thiols, amines, amides, thiols, ethers, esters, carbonyls, etc.). The functional groups may have desirable resonance structures. The polyols solubilize polar to modestly polar compounds. It is desirable that the polyol is non-toxic (e.g., the polyol has an LD₅₀ (rat/oral) of greater than 300). It may be desirable to use a polyol or polyols that have an LD₅₀ three to five times greater than the aforementioned 300. Combinations of one or more polyols can be used. The polyol(s) is/are present in the composition at 25 to 98 mol %, including all integer mol % values and ranges therebetween.

Examples of suitable polyols include 1,3-butanediol (e.g., racemic), 1,2-butanediol (e.g., racemic), 2,3-butanediol (e.g., racemic), 1,2,5-pentanetriol (e.g., racemic), 1,3,5-pentanetriol (e.g., racemic), 2,4-pentanediol (e.g., racemic), 1,2-pentanediol (e.g., racemic), and 1,2,6-hexanetriol (e.g., racemic). Based in part on their low toxicity (LD₅₀), the polyols serve as desirable, low-boiling-point alternatives to propylene glycol. Further, their characteristics also serve to offset the need for 1,2,3-propanetriol as a co-excipient commonly found in vaping excipients. In an embodiment, the compositions do not solely comprise propane-1,2-diol and/or 1,2,3-propanetriol. In another embodiment, the compositions do not comprise propane-1,2-diol and/or 1,2,3-propanetriol. Polyols are commercially available or can be manufactured using methods known in the art.

In various embodiments, the polyol is a tetraol (e.g., (2R, 3S) butane-1,2,3,4-tetraol (erythritol) and (2R,3R)-butane-1,2,3,4-tetraol (threitol)), a pentol (e.g., (2R,4R)-pentane-1,2,3,4,5-pentol (arabitol), (2R,4S)-pentane-1,2,3,4,5-pentol (xylitol), (2R,3S,4S)-pentane-1,2,3,4,5-pentol (ribitol), and (2R,3S,4R,5S)-hexane-1,2,3,4,5-pentol (fucitol)), a hexol (e.g., (2R,3R,4R,5R)-hexan-1,2,3,4,5,6-hexol (mannitol), (2S,3R,4R,5R)-hexane-1,2,3,4,5,6-hexol (sorbitol), (2R,3S,4R,5S)-hexane-1,2,3,4,5,6-hexol (galactitol), and (2R,3S,4S,5R)-hexane-1,2,3,4,5,6-hexol “iditol”)), a heptol (e.g., (2R,3R,5R,6R)-heptane-1,2,3,4,5,6,7-heptol (volemitol) and (2S,3S,5S,6S)-heptane-1,2,3,4,5,6,7-heptol (perseitol)).

In an embodiment, the polyol is a polyol ether. Examples of suitable polyol ethers include isomalt ((2R,3R,4R,5R)-6-[[(2S,3R,4S,5S,6R)-3,4,5-trihydroxy-6-(hydroxymethyl)-2-tetrahydropyranyl]oxy]hexane-1,2,3,4,5-pentol), lactitol (4-O-α-D-galactopyranosyl-D-glucitol), and maltitol (4-O-α-D-glucopyranosyl-D-glucitol).

The composition may comprise disruptive compounds. A variety of disruptive compounds can be used in the compositions. Without intending to be bound by any particular theory, it is considered that addition of one or more disruptive compounds introduces a slightly disruptive property to the systems, thereby lowering the viscosity and/or the boiling point (being a contributing factor to the overall positive azeotropic system) while serving as a solubilizing factor, due to hydrogen bonding, resulting in the compound forming an azeotrope, or acting as a buffer, chaotrope, hydrotrope, kosmotrope, surfactant, cage molecule, or chelating agent. Combinations of disruptive compounds can be used. For example, the disruptive compound(s) are present at 0 to 8 mol %, including all integer mol % values therebetween. In an embodiment, the disruptive compound(s) are present at 1 to 8 mol %. Suitable disruptive compounds are commercially available or can be manufactured using methods known in the art.

Examples of suitable disruptive compounds include a bases (e.g., a base such as MOH, M₂CO₃ (M=Na, K) (e.g., adding a 0.001-0.075M solution of such a base)), C₁ to C₆ linear aliphatic alcohols with a single hydroxyl group (e.g., ethanol, propanol, or 2-methyl-2-butanol), linear carbohydrates, chelating compounds (e.g., tetramethylethylenediamine (TMEDA)), cage molecules (e.g., cyclodextrins), hydrotropes (e.g., sodium benzoate), chaotropes (e.g., urea), kosmotropes (e.g., trehalose), and surfactants (e.g., phospholipids).

Examples of suitable linear carbohydrates include trioses, such as mannitol and xylitol. Without intending to be bound by any particular theory, it is considered that short-chain linear sugars lower viscosity of the composition, due to their librative properties. This viscosity lowering effect was particularly evidenced when a linear carbohydrate or mixture of such carbohydrates was used in conjunction with ethanol. The linear carbohydrates solubilize active molecules with both polar moieties and non-polar backbones/chains (e.g., biotin, folic acid, and pyridoxine). For example, a composition comprises 1% biotin with the remainder being mannitol to cite merely one example, also demonstrated efficacy in serving as a “bridging adduct.”

Examples of suitable cage molecules include α-, β-, and γ-alkoxy-cyclodextrins. α-, β-, and γ-alkoxy-cyclodextrins were successfully used in equimolar concentrations with the target, i.e., the non-polar molecule to be solubilized (e.g., vitamin D3). For example, the compounds are first mechanically mixed and then a 0.1-0.4 mole amount of any n-unsaturated fatty acid (2≤n≤4), such as safflower, hemp, flaxseed, citronella, linoleic, linolenic, or a combination thereof, was added. Then, heat (e.g., 135-155° C.) is carefully, intermittently applied to the system (e.g., for 40-50 seconds). In an alternative example, a 3:1 molar ratio of target to fatty acid was prepared first until dissolution. Then, this material was pipetted into a receiving solution with an equimolar amount of (α-, β- or γ-) alkoxy-cyclodextrin dissolved in the hydroxylated compositions described above. Alternatively, seven low-wattage (150 W) microwave, 30 second bursts were applied with gentle, intermittent agitation to the mechanically mixed solution.

The disruptive compounds may also have additional properties. For example, the disruptive compound is also a flavorant (e.g., isoprene is both a disruptive compound and flavorant).

A variety of active compounds can be used in the compositions. The active compounds are compounds that provide a benefit to an individual to which the active compound is delivered in the vapor phase. The compounds are polar or non-polar compounds or have polar and/or non-polar domains. In an embodiment, the active compound(s) is/are free-base crystals and are not adducts (e.g., complex salts). Combinations of active compounds can be used. Suitable active compounds are commercially available or can be manufactured using methods known in the art.

Examples of suitable active compounds include vitamins and their vitamers, and derivatives thereof (e.g., vitamin A compounds, such as, for example, retinol, beta-carotene, alpha-carotene, gamma-carotene, beta-cryptoxanthin, vitamin B compounds, such as, for example, thiamine, riboflavin, niacin, niacinamide, pantothenic acid, pyridoxine, biotin, folate, and cyanocobalamin, vitamin C compounds, such as, for example, ascorbic acid, vitamin D compounds, such as, for example, vitamins D1, D2, D3, D4, and D5, vitamin E compounds, such as, for example, α-tocopherol, tocotrienol, and γ-tocopherol, and vitamin K compounds, such as, for example, K1 and K2); antioxidants (such as, for example, trans resveratrol, n-acetylcysteine); hormones (such as, for example, melatonin, insulin (e.g., insulin hexamer), estradiol, testosterone, and oxytosin); diarylheptanoids (such as, for example, curcumin and demethoxycurcumin); zwitterionic dipeptides (such as, for example, carnosine and acetyl carnosine); flavonoids (such as, for example, flavan-3-ol, catechin, epicatechin gallate, myricetin and gallocatechol); phenylethanoids (such as, for example, tyrosol, hydroxytyrosol, and acetyl hydroxytyrosol); polyphenols; benzopyrones; naturally occurring phenols; flavanols; procyanidins (such as, for example, kaempferol, galangin, carbenoxolone, ferulic acid, caffeic acid phenethyl ester, fraxetin, echinacea alkamides, Co Q10), tyrosol, and curcumin); carotenoids and xanthophils (such as, for example, α-δ carotene; β-cryptoxanthin, neoxanthin); phenylpropanoids and glycosides (such as, for example, echinacoside, caffeic acid glycoside); nootropic compounds (such as, for example, carnitine, acetyl carnitine, choline, cytidine phosphate choline, CDP choline, alpha GPC, phosphatidylcholine, amino acids and their derivatives (e.g., tyrosine and acetyl 1-tyrosine), modafinil, adrafinil, and armodafinil); phospholipids (such as, for example, phosphatidylserine, phosphatidylcholine); omega-3-fatty acids (such as, for example, DHA, EPA, and alpha linolenic acid); racetams (such as, for example, piracetam, aniracetam, oxiracetam, phenylpiracetam, fasoracetam, coluracetam, and pramiracetam); tianeptine; peptides (such as, for example, noöpept, selank, acetyl selank, semax, acetyl semax, and fabomotizole); 5-HTP; l-theanine; purines and nitrogenous bases (such as, for example, caffeine, phenibut; picamilon; pyritinol; sulbutiamine,); alkaloids and alkaloid derivatives (such as, for example, huperzine A, vinpocetine and vincamine); uridine monophosphate; immunodilators (such as, for example, zinc complexes (e.g., complexes with monodentate and/or bidentate ligands) such as, for example, zinc citrate), β-glucan, arginine, l-theanine); pure, freebase crystals (so-called “active compounds”) derived from (e.g., extracted from) plants or extracts (such as, for example, Lion's Mane Mushroom, Rhodiola Rosea, Cordyceps, Garlic, Astralagus, St. John's Wort, Milk Thistle, Ginger, Ginseng, Echinacea, Eleuthero, Ashwagandha, Dangshen and similar plant species); carboxylic acids (such as, for example, ACE inhibitors), synthetic hydroxymethylphenols (such as, for example, albuterol), sulfonamides or amids in general such as diuril, nialamide, furosemide or torsemide, methyl-n-oates or heptanoic acids (such as, for example, lovastatin or atorvastatin, respectively), sulfonamides or amide (such as, for example, diuril, nialamide, furosemide or torsemide), conjugate bases (such as, for example, dicarboxylates (e.g., amlodipine and other calcium-channel blockers), and drugs (such as, for example, sleep aids, pain killers, mucokinetics, antidepressants, HIV drugs and decongestants, including for example, selegiline, guaifenesin, tyloxapol, acetylcysteine, dextromethorphan, diphenhydramine, loratidine, phenylephrine HCl, alkaloids and alkaloid purines (such as, for example, theophylline, berberine, piperine, chelerythrine, quinidine, synribo, and ephedrine), fucoxanthin, fenugreek, hordenine, and sildenafil (Viagra™)). Additional examples of active compounds include mannitol, xylitol, and trigonelline.

The compositions may comprise one or more solubilizing compounds (also referred to herein as co-solute compounds). The solubilizing compounds may also be active compounds. The solubilizing compounds are polar (e.g., modestly polar compounds), non-polar, or complex non-polar compounds that increase the solubility of the one or more active compounds in the composition. The solubilizing compounds may be planar and have both polar and nonpolar regions that reduce hydrophobic interactions. Without intending to be bound by any particular theory, it is considered that the solubilizing compound forms adducts with the polyol(s) and/or disruptive compound(s). Compositions comprising on or more solubilizing compounds may form hydrotropes. Combinations of solubilizing compounds may be used in the compositions. The solubilizing compounds may be present in the compositions at 1 to 40 mol %, including all integer mol % values and ranges therebetween.

Examples of suitable solubilizing compounds include amino acids and derivatives thereof, peptides, vitamins and derivatives thereof, zwitterionic compounds, amidoglycans, short-chain polyol carboxylic acids (e.g., hyaluronic acid n-mers (n=1-4)), isoprenes, xanthines, purines, lactones, lactams, heterocyclic compounds, phenolic compounds, pyridines, ketones, esters, phospholipids, quarternary ammonium salts. For example, the solubilizing compounds are used to increase the solubility of polar or “complex non-polar” molecules include biotin, folic acid, complex xanthines, zwitterions with complex, extended backbones between the zwitterionic moieties).

Additional examples of suitable solubilizing compounds include vitamins (e.g., niacinamide, niacin, thiamine, and biotin) and their vitamers/derivatives (e.g., nicotinic acid and N-allylnicotinamide), pyridines (e.g., piperazine), benzene diols (e.g., catechol, resorcinol, and pyrogallol), phenolic compounds (e.g., guaiacol, acetyl hydroxytyrosol), hyroxybenzoic acids (e.g., salicylic acid), heterocyclic sugars (e.g., isosorbide), organic acids (e.g., taurine, citric acid), amino acids (e.g., proline, valine, leucine, isoleucine, alanine, and arginine as well as their derivatives e.g., glycine and trimethylglycine), chelating agents (e.g. citric acid), niosomes, hydrotropic salts (e.g., sodium benzoate, and sodium salicylate), carbonate esters (e.g., ethylene carbonate, propylene carbonate), dimethyl ethers (e.g., syringol), imidazoles (e.g., lysidine), nucleosides (e.g., guanosine), nitrogenous organic compounds (e.g., creatine, creatine methyl ester, methylarginine, octopine, phosphocreatine, glycocyamine, guanidinopropionic acid), monosaccharides (e.g., glucose, fructose, galactose), disaccharides (e.g., trehalose), polysaccharides (e.g., β-glucan), tetrose (e.g., erthrose), pentose (e.g., ribose), hexose (e.g., allose), lactams (e.g., caprolactam), carboxylic acids (e.g., lipoic acid), dicarboxylic acids (e.g., mallic acid), tricarboxylic acids (e.g., citric acid), chelating agents (e.g., dimercaptosuccinic acid), cyclic esters (e.g., lactide), alkaloids (e.g., hurperzine A), alkaloidal amines (e.g., sinapine), phytochemicals (e.g., monolignols), organosulfur compounds (e.g., sulfolane, taurine), zwitterions (e.g., lysine), betaines (e.g., trimethyl glycine, a glycerylphosphorylcholine), benzopyrones (e.g., fraxetin), cyclic compounds (e.g., phytic acid, inositol nicotinate), phospholipids (e.g., phosphatidylethanolamine, phosphatidylglycerol, phosphatidylinositol, phosphoinositide), quarternary ammonium salts and their derivatives (e.g., choline, phosphatidyl choline, cytidine diphosphate-choline), amino sugars (e.g., glucosame, sialic acid), imino sugar (e.g., swainsonine), macrocycles (e.g., a cyclodextrin, β cyclodextrin, γ cyclodextrin, porphin, chlorin, corrin), alkyl glyceryl ethers (e.g., 1-methylbutyl α-glyceryl ether), and aryl glyceryl ethers (e.g., benzyl α-glyceryl ether).

The compositions may comprise one or more additional compounds. The additional compounds may also increase the solubility of the active compounds. Examples of additional compounds include flavorants (e.g., carvone, ethyl vanillin, isopurines, cinnamic acid, sugars, etc.), preservatives (e.g., antioxidants such as sodium benzoate), colorants, pH modifiers, stabilizing compounds, viscosity modifiers, and emulsifiers. In various embodiments, the additional compound(s) is/are a food additive or food additives with an E Number (following the International Numbering System (INS)) of E100 to E1599.

The compositions may comprise water. Water is a reasonably high dipole-moment molecule that serves as hydrogen-bonding adduct. The water may make up the remainder of a composition. For example, the water is present in the composition at up to 65 volume percent. In an embodiment, the composition comprises 0 to 65 volume percent water, including all integer volume percent values and ranges therebetween.

In an embodiment, the polyols are glycerin and 2,3-butanediol, the disruptive compound is niacinamide, the active compound is vitamin B12, and optionally included are additional compounds (ethanol and/or various esters). In an embodiment, the polyols are glycerin and 2,3-butanediol, the disruptive compounds are ethanol, biotin, and niacinamide, and the active compound is folic acid. In an embodiment, the polyols are glycerin and 2,3-butanediol, the disruptive compound is β-cyclodextrin, the active compound is vitamin D3, and the additional compounds are cinnamic acid and ethyl vanillin. Water is an optional component of all of these embodiments. Additionally, these embodiments may include any of the other optional components disclosed herein.

In an embodiment, the polyols are 2,3 butane diol (e.g., 30-70% v/v) 48-49.5% v/v or 48-62% v/v) and 1,2,3-propanetriol (e.g., 33-70% v/v or 48-49.5% v/v). In another embodiment, the optional disrupting compound in this embodiment is ethanol (e.g., 1-5% v/v).

In an embodiment, a composition comprises racemic 2,3 butane diol (e.g., 48-49.5% v/v), 1,2,3-propanetriol (e.g., 48-49.5% v/v), ethanol (e.g., 1-4% v/v), one or more active compound, and, optionally, one or more disrupting compound. In another embodiment, a composition comprises 1,2,3-propanetriol (e.g., 62% v/v), racemic 2,3 butane diol (e.g., 33% v/v), ethanol (e.g., 5% v/v), one or more active compound, and, optionally, one or more disrupting compound. In another embodiment, a composition comprises 1,2,3-propanetriol (e.g., 70% v/v), racemic 2,3 butane diol (e.g., 30% v/v), ethanol, one or more active compound, and, optionally, one or more disrupting compound. In another embodiment, a composition comprises 1,2,3-propane triol (e.g., 30% v/v), racemic 2,3 butane diol (e.g., 67.5% v/v), ethanol (e.g., 2-3% v/v), one or more active compound, and, optionally, one or more disrupting compound.

In an aspect, the present disclosure provides methods for making the compositions. The compositions can be made by mixing together the individual components (e.g., polyol(s), optionally, disruptive compounds(s), active compound(s), optionally, water, and any other optional components). The individual components can be combined in any order. The compositions may be heated at any point during the mixing of the components or after the components are mixed together.

Typically, the liquid or solution components (e.g., polyol(s) and disruptive compound(s)), if present, are mixed into the dry (i.e., solid) components (e.g., polyol(s) and disruptive compound(s)), if present. Then solvent(s) may be added. The mixture may be stirred and/or heated (e.g., thermally heated and/or microwave heated. In an example, the mixture is thermally heated and/or microwave heating at 30 s bursts with 30 s pauses at 100-200 W). Then, the active component(s) are added. The active component(s) may be added gradually, in parts. Solid components are typically added gradually to the liquid, heated appropriately, and then stirred until dissolved. For example, no more 45 mg of solid component is added/ml of solution in each increment. When adding another liquid component, vigorous shaking may be required.

In an aspect, the present disclosure provides methods of using the compositions. For example, the compositions are used in methods of administering active compound(s) in the vapor phase to an individual. The administration to the individual is via the transalveolar tissue passage of the individual and/or the mucosal lining of the airway of the individual. The administration may result in at least a detectible amount of the active compound(s) and/or its/their derivative(s) (e.g., metabolite) in the blood stream (e.g., serum and/or blood plasma) and/or urine and/or central nervous system (CNS) fluid of the individual. In an embodiment, the active compound(s) is/are not administered to the individual as an aerosol.

The active compound(s) and/or its/their derivative(s) can be detected by methods known in the art. For example, the active compound(s) and/or its/their derivative(s) can be detected using enzyme-linked immunosorbent assay (ELISA), spectroscopic analysis, and/or chromatographic methods (e.g., gas chromatography or liquid chromatography). In various examples, the active compound(s) and/or its/their derivative(s) are detected by analysis of blood, serum, or urine (e.g., urinalysis).

In an embodiment, a method of vapor phase delivery of an active compound or active compounds comprises administering an active compound or active compounds in the vapor phase to an individual using the composition of the present disclosure.

The compounds can be administered to a variety of individuals. For example, the individual is a human or non-human animal (e.g., non-human mammal).

A variety of apparatuses can be used to administer the active compound(s) to an individual. The apparatus administers the active compound(s) to the individual via the transalveolar tissue passage of the individual and/or the mucosal lining of the airway of the individual. The apparatus may be a wicked (draws composition from a reservoir by capillary action) or wickless apparatus (the resistive coil is in liquid contact with the composition). It is desirable to use a low power (e.g., 7-12 W (low resistance (0.5-1.2 Ohms)/low voltage (e.g., 2-3 V) DC circuit. It is also desirable to minimize coking or have no coking, which may be realized by using a low wattage/low voltage apparatus. Examples of suitable apparatuses include vaporizing apparatuses such as electronic vaporizing apparatuses. Suitable apparatuses are known in the art. Examples of suitable apparatuses include Clearomizer and MVE Carbo-Mizer®.

For example, the vaporizing device is any device that can store the composition in a reservoir and has a wick in direct or proximate contact with the composition and the wick is in direct contact with a resistive coil. At or near the moment of the individual's inspiration, either the act of inspiration or inspiration with a toggle button triggers the closure of a circuit including the resistive coil, thereby heating the coil and rendering the active compound, which is in the liquid state in the composition, into the vapor state and is administered to the individual.

The active compound(s) retain at least a portion of its/their activity (i.e., benefit to the individual) after delivery to the individual. In an embodiment, the active compound(s) retain all or substantially all of its/their structure and/or activity after administration to the individual. By “substantially all” it is meant that the active compound(s) retain 50% or more of its/their structure and/or activity after administration to the individual. In various embodiments, the active compound(s) retain 60% or more, 70% or more, 80% or more, 90% or more, or 95% or more of its/their structure and/or activity after administration to the individual. The structure and/or activity of the active compound(s) in the individual can be determined by methods known in the art.

The steps of the methods of making compositions of the present disclosure and methods of administering compositions of the present disclosure described in the various embodiments and examples disclosed herein are sufficient to carry out methods of making compositions of the present disclosure or methods of administering compositions of the present disclosure. Thus, in an embodiment, a method of making a composition of the present disclosure or a method of administering a composition of the present disclosure consists essentially of a combination of the steps of the methods disclosed herein. In another embodiment, a method of making a composition of the present disclosure or a method of administering a composition of the present disclosure consists of such steps.

In an aspect, the present disclosure provides kits. A kit can comprise one or more compound of the present disclosure and instructions for its use. Optionally, a kit includes a vaporizing apparatus (e.g., an electronic vaporizing apparatus).

In the following Statements, various examples of the compositions, methods, and kits of the present disclosure are described:

Statement 1. A composition for vapor phase delivery of active compounds comprising: a) one or more polyol of the present disclosure; b) optionally, one or more disruptive compound of the present disclsoure; and c) one or more active compound of the present disclosure.
Statement 2. A composition according to Statement 1, wherein the composition further comprises a solubilizing compound (e.g., one or more solubilizing compound of the present disclosure).
Statement 3. A composition according to any one of the preceding Statements, wherein the composition further comprises an additional compound (e.g., one or more additional compound of the present disclosure).
Statement 4. A composition according to any one of the preceding Statements, wherein the composition further comprises an organic solvent (e.g., one or more organic solvent of the present disclosure) and/or water.
Statement 5. A composition according to any one of the preceding Statements, wherein the polyol is selected from the group consisting of diols, triols, tetraols, pentaols, hexols, heptols, polyol ethers, and combinations thereof.
Statement 6. A composition according to any one of the preceding Statements, wherein the polyol is selected from the group consisting of 1,3-butanediol, 1,2-butanediol, 2,3-butanediol, 1,2,5-pentanetriol, 1,3,5-pentanetriol, 2,4-pentanediol, 1,2-pentanediol, 1,2,6-hexanetriol, 1,2,3-propanetriol, (2R, 3S) butane-1,2,3,4-tetraol, (2R,3R)-butane-1,2,3,4-tetraol, (2R,4R)-pentane-1,2,3,4,5-pentol. (2R,4S)-pentane-1,2,3,4,5-pentol, (2R,3S,4S)-pentane-1,2,3,4,5-pentol, and (2R,3S,4R,5S)-hexane-1,2,3,4,5-pentol, (2R,3R,4R,5R)-hexan-1,2,3,4,5,6-hexol, (2S,3R,4R,5R)-hexane-1,2,3,4,5,6-hexol, (2R,3S,4R,5S)-hexane-1,2,3,4,5,6-hexol, and (2R,3S,4S,5R)-hexane-1,2,3,4,5,6-hexol, (2R,3R,5R,6R)-heptane-1,2,3,4,5,6,7-heptol, (2S,3S,5S,6S)-heptane-1,2,3,4,5,6,7-heptol, ((2R,3R,4R,5R)-6-[[(2S,3R,4S,5S,6R)-3,4,5-trihydroxy-6-(hydroxymethyl)-2-tetrahydropyranyl]oxy]hexane-1,2,3,4,5-pentol), (4-O-α-D-galactopyranosyl-D-glucitol), (4-O-α-D-glucopyranosyl-D-glucitol), and combinations thereof.
Statement 7. A composition according to any one of the preceding Statements, wherein the polyol is racemic 2,3 butane diol and/or 1,2,3-propanetriol.
Statement 8. A composition according to any one of the preceding Statements, wherein the disrupting compound is selected from the group consisting of bases, C₁ to C₆ linear aliphatic alcohols with a single hydroxyl group, linear carbohydrates, chelating compounds, cage molecules, hydrotropes, chaotropes, kosmotropes, surfactants, and combinations thereof.
Statement 9. A composition according to any one of the preceding Statements, wherein the disrupting compound is ethanol.
Statement 10. A composition according to any one of the preceding Statements, wherein the active compound is selected from the group consisting of vitamins and their vitamers, and derivatives thereof, antioxidants, hormones, diarylheptanoids, zwitterionic dipeptides, flavonoids, phenylethanoids, polyphenols, benzopyrones, naturally occurring phenols, flavanols, procyanidins, carotenoids, xanthophils, phenylpropanoids, glycosides, amino acids and their derivatives, phospholipids; omega-3-fatty acids; racetams; peptides; purines, nitrogenous bases, alkaloids, alkaloid derivatives, immunodilators, pure, freebase crystals derived from plants, plant extracts; carboxylic acids, ACE inhibitors, synthetic hydroxymethylphenols, sulfonamides, amides, methyl-n-oates, heptanoic acids, sulfonamides or amides, conjugate bases, drugs, and combinations thereof.
Statement 11. A composition according to any one of the preceding Statements, wherein the active compound is selected from the group consisting of retinol, beta-carotene, alpha-carotene, gamma-carotene, beta-cryptoxanthin, thiamine, riboflavin, niacin, niacinamide, pantothenic acid, pyridoxine, biotin, folate, cyanocobalamin, ascorbic acid, vitamin D1, vitamin D2, vitamin D3, vitamin D4, vitamin D5, α-tocopherol, tocotrienol, γ-tocopherol, vitamin K1, vitamin K2, trans resveratrol, n-acetylcysteine, melatonin, insulin, insulin hexamer, estradiol, testosterone, oxytosin, curcumin and demethoxycurcumin, carnosine, acetyl carnosine, flavan-3-ol, catechin, epicatechin gallate, myricetin, gallocatechol, tyrosol, hydroxytyrosol, acetyl hydroxytyrosol, kaempferol, galangin, carbenoxolone, ferulic acid, caffeic acid phenethyl ester, fraxetin, echinacea alkamides, Co Q10, tyrosol, and curcumin), α-δ carotene, β-cryptoxanthin, neoxanthin, echinacoside, caffeic acid glycoside, carnitine, acetyl carnitine, choline, cytidine phosphate choline, CDP choline, alpha GPC, phosphatidylcholine, tyrosine and acetyl 1-tyrosine, modafinil, adrafinil, armodafinil, phosphatidylserine, phosphatidylcholine, DHA, EPA, alpha linolenic acid, piracetam, aniracetam, oxiracetam, phenylpiracetam, fasoracetam, coluracetam, pramiracetam, tianeptine, noöpept, selank, acetyl selank, semax, acetyl semax, and fabomotizole, 5-HTP, l-theanine, caffeine, phenibut; picamilon; pyritinol; sulbutiamine, huperzine A, vinpocetine, vincamine, uridine monophosphate, zinc citrate), β-glucan, arginine, l-theanine; Lion's Mane Mushroom, Rhodiola Rosea, Cordyceps, Garlic, Astralagus, St. John's Wort, Milk Thistle, Ginger, Ginseng, Echinacea, Eleuthero, Ashwagandha, Dangshen, albuterol, diuril, nialamide, furosemide, torsemide, lovastatin, atorvastatin, amlodipine, selegiline, guaifenesin, tyloxapol, acetylcysteine, dextromethorphan, diphenhydramine, loratidine, phenylephrine HCl, theophylline, berberine, piperine, chelerythrine, quinidine, synribo, ephedrine, fucoxanthin, fenugreek, hordenine, sildenafil, mannitol, xylitol, trigonelline, and combinations thereof.
Statement 12. A method for vapor phase delivery of an active compound or compounds comprising: administering (e.g., using administration disclosed herein) an active compound or active compounds of the present disclosure in the vapor phase to an individual using a composition of the present disclosure (e.g., a composition of any one of Statements 1 to 11).
Statement 13. A method according to Statement 12, wherein after administration of the compound the individual has a detectible amount of the active compound(s) or a derivative of the active compound(s) (e.g., one or more metabolite(s)) in its blood and/or urine.
Statement 14. A method according to any one of Statements 12 or 13, wherein the active compound is rendered into the vapor phase using a vaporizing apparatus (e.g., a vaporizing apparatus of the present disclosure).
Statement 15. A method of Statement 14, wherein the vaporizing apparatus is an electronic vaporizing apparatus (e.g., an electronic vaporizing apparatus of the present disclosure).
Statement 16. A kit comprising: a) one or more compositions of the present disclosure (e.g., one or more compositions of any one of Statements 1 to 11); and b) instructions for use of the one or more compositions.
Statement 17. A kit according to Statement 16, further comprising a vaporizing apparatus (e.g., a vaporizing apparatus of the present disclosure).
Statement 18. A kit according to any one of Statements 16 or 17, wherein the vaporizing apparatus is an electronic apparatus (e.g., an electronic vaporizing apparatus of the present disclosure).
Statement 19. A kit according to any one of Statements 16 to 18, wherein the instructions describe how to administer the one or more compositions to an individual.

The following examples are presented to illustrate the present disclosure. They are not intended to limiting in any manner.

### Example 1

The following provides examples of compositions of the present disclosure and making the compositions.

Folate. Folate and biotin are the two most difficult to dissolve B vitamins. Folate solubility enhancement was provided by addition of beta-cyclodextrin. A clear solution of folate and beta-cyclodextrin was formed without heating when combined with 1% biotin/99% mannitol (filler) powder. Previous experiments using beta-cyclodextrin to dissolve folate required heating but the following solution: 2.5 ml stock*+0.15 ml water+0.15 ml ethanol+0.00001 mole beta-cyclodextrin [0.01135 g]+0.0001 mole of combined biotin/mannitol [0.0182 g]+0.00001 mole folate [0.00441]) was clear with some orange (folate) precipitate on the bottom of the vial. *stock refers to the 70:30 mole mixture of glycerin and 2,3 butane diol

The solubility enhancement from mannitol/biotin alone was tested next. It was found that a solution of 3 ml stock+0.25 ml water+0.25 ml ethanol+0.0182 g of biotin/mannitol) was capable of dissolving approximately 0.008 g of folate (8 mg) when the solution was prepared with the procedure outlined below. This is major improvement over most polar solvents (0.0016 g/L of water at 25° C.).

First the mannitol/biotin powder was mixed with 0.004 g of folate. This was added to the test tube followed by the liquid. It was stirred vigorously. Then it was heated with a heat gun until clear and no folate was suspended. The next day, no orange/red precipitate was found, indicating that only the mannitol/biotin had not dissolved. All the liquid was moved to another test tube and the precipitate remained. Then folate was incrementally added (about 1 mg) and the solution was stirred, followed by heating** until it became clear. It would take at least 3 minutes of heating for it to become clear but as more folate was added, at least 4 minutes of heating were needed. This is how the original solution was formed. Over the course of several days there was no further precipitate or increase in viscosity. ** all heating was done with the yellow heat gun on the lowest setting (240° F.)

Upon repeating this experiment, the solution was found to be even more effective at dissolving folate. While heating and stirring the lid popped off and 0.75 ml of solution spilled out. Even accounting for folate lost in the spill, currently at least 8 mg of folate have been dissolved in 2.75 ml and the solution is completely clear. Heating the solution caused the lid to pop so it must be removed after about 1 minute and 30 seconds of heating prior to any further heating. It is also crucial for the solution to have been flipped upside down, preferably while warm. The solution must not be heated while flipped as this may cause a spill.

The next day it was found that the 2nd solution turned clear orange and was extremely viscous. The original solution still has less much viscosity but also a slightly lower concentration of folate. It appears that somewhere beyond 8 mg folate/3.5 ml of solution it becomes supersaturated, resulting in a thick gel upon settling and cooling. However heating the thickened solution quickly (just over 1 minute) changed its color back to yellow and it had a similar viscosity as the original solution. As long as supersaturation followed by settling is avoided, the solution will remain liquid. Another option is to study the effect of precipitation inhibitors or the sonicator to further boost solubility without increasing viscosity.

Testing was also done with only stock, stock+water and stock+ethanol but having both water and ethanol is crucial to the process. Furthermore it was found that not using mannitol/biotin powder at all was ineffective in dissolving folate (<1.6 mg) in the liquid only part of the same solution. Despite having separated the liquid from the precipitate which was nearly all biotin/mannitol, the powder still played a role in improving folate solubility. The next round of experimentation was done with pure 100% biotin which appears to be even more effective in dissolving folate. However biotin has low solubility in in the solvent (about 3 mg/ml). The following composition (3 ml stock+0.25 ml water+0.25 ml ethanol+0.009 g of biotin) dissolved 11.2 mg of folate but became extremely viscous.

According to research on drug solubility there is a class of molecules known as hydrotropes (also known as chaotropes, salting out). They reduce hydrophobic forces between non-polar solutes in polar solvents. One such molecule is niacinamide (vitamin B3 amide). These hydrotropes are planar and are either very polar or have nonpolar and polar sides like surfactants but shorter. This experiment was repeated with niacinamide as a cosolute and with a combination of B3 amide and Biotin. The biotin and B3 amide were added along with 4 mg of folate (and crushed together) wet into dry to the solution. The remaining folate was added incrementally along with heating and stirring. Ultimately the combination of B3 was found to be the most successful at dissolving folate and was also necessary to lower the viscosity of the solution. (2.5 ml stock+0.5 ml water+0.5 ml ethanol+0.0221 g of B3 amide) dissolved approximately 11 mg of folate and a small amount of precipitate. (2.5 ml stock+0.5 ml water+0.5 ml ethanol+0.0221 g of B3 amide+0.090 g pure biotin) dissolved approximately 11.4 mg of folate without precipitate. Unfortunately both solutions were extremely viscous after a few days.

Eventually the B3 amide and biotin cosolute solution was prepared using “reverse stock” instead of regular with the goal of lowering viscosity. The reverse stock is a solution of 3:7 mol glycerin and 2,3 butane diol. Initially this made folate precipitate so additional glycerin was added to achieve a 1:1 mol ratio of glycerin to 2,3 butane diol. 9 mg of folate was found to dissolve in the following: 0.0221 g of B3 amide, 0.0047 mg biotin, 3.5 ml of equimolar glycerin and 2,3 butane diol, 0.5 ml of water, 0.5 ml of ethanol. The following flavors were added without affecting the solubility of the folate or the viscosity: 0.1 ml of carvone, 0.125 ml of flavored vodka (35% ethanol, 65% water, and additional esters for flavoring) and 0.125 ml of flavored glycerin (glycerin infused with esters).

Trans Resveratrol—Trans-resveratrol (referred to as TR) is a potent antioxidant with a very non polar molecular structure and very low solubility in water (0.03 g/L) but higher solubility in other solvents (50 g/L in ethanol). The initial approach was to test non polar solvents and their effectiveness with ethanol. This included pure stock as a control, carvone, and combinations of those solvents with ethanol. The following solvents were prepared: Control—3.5 ml of pure stock, 1-1.75 ml of stock+1.75 ml of carvone+0.2 ml of 0.01 mol NaOH, 2-3.5 ml of pure carvone, 3-3 ml of stock+0.5 ml of ethanol, 4-3 ml of carvone+0.5 ml of ethanol.

Solutions 2 and 4 easily dissolved the folate without needing any heating. Solution 1 became biphasic and cloudy. The control and solution 4 have approximately the same solubility of TR. Unlike folate, TR takes longer time to precipitate so I kept adding TR until it was greatly oversaturated (0.1 g/ml) and this solution had a great deal of precipitate 2 days later. Solvent was added until the TR was dissolved which occurred at 60 mg/ml of stock. This original solution was split into 5 test tubes of 1 ml each with about 0.06 g of TR.

The following solutions were made: 1—adding 0.47 ml of carvone (equimolar with TR, 0.000263 mol of each) 2—adding 0.175 ml of ethanol 3-0.235 ml of carvone and 0.1 ml of ethanol. Solution 1 became cloudy because the carvone is immiscible with the stock. Several attempts to fix this were made using of ethanol and B3 amide as hydrotropes but it had no effect. Solution 2 still had a solubility of 0.06 g/ml.

Next co-solutes were used along with the TR in stock solution. 0.000263 mol of the following were dissolved along with TR in 3 ml stock: 1-niacin, 2-caffeine, 3-gallic acid, 4-salicylic acid, 5-thiamine, 6-niacinamide, and 7-pantothenic acid. Two additional samples were prepared of 0.01 g biotin (8) and 0.04 g of -pyridoxine HCl (9). Each solution consisted of the stated amount of co-solute and 3 ml of stock. The following amounts of TR (which was added incrementally (30-40 mg) and heated for 3-4 minutes by heat gun) created the following:

1—0.1776 g, slight visible suspension of particles
2—0.0131 g, immediately precipitated due to low solubility of caffeine in pure stock solution
3—0.1784 g, clearly visible suspension and precipitate on bottom
4—0.1784 g, small amount of suspension and precipitate are visible in solution
5—0.1808 g, small amount of suspension and precipitate are visible in solution
6—0.1738 g, totally clear solution with low viscosity
7—0.0131 g, cloudy solution without precipitate
8—0.1733 g, tiny amount of suspension
9—0.1859 g, clearly visible suspension with precipitate

The hypothesis is that B3 amide worked better than B3 because it has a more polar functional group (amide>carboxylic acid) on one side. The other side is simply a benzene ring with a nitrogen atom. The same applies for gallic vs salicylic acid, which have a very similar structure but gallic acid has 3 hydroxyl groups on the opposite side of the carboxylic acid which makes it more polar. This will be useful when adding flavor to the final TR composition. Esters and any flavor compounds which are nonpolar except for a carboxylic acid (such as but not limited to cinnamic acid and ethyl vannilin or their derivatives) would be more suited for flavors. Sugars may work as well.

To conduct further testing 0.5 ml of ethanol was added to the solutions with niacin, biotin, thiamine, and salicylic acid. The successful B3 amide solution was split into two solutions of (1.625 and 1.375 ml) with 0.25 ml of ethanol being added to the 1.375 ml solution. The results were as follows:

0.0698 g B1+3.5 ml stock+0.5 ml ethanol: 0.2333 g TR
0.0324 g B3+3.5 ml stock+0.5 ml ethanol: 0.2316 g TR
0.0100 g B7+3.5 ml stock+0.5 ml ethanol: 0.2606 g TR
0.048175 B3 amide+1.625 ml and 0.048175 B3 amide+1.375+0.25 ml ethanol both dissolved 0.125 g TR/ml.

There was also testing with multiple solutes in 3 ml of stock:

0.0482 g B3 amide+0.03 g B1 dissolved 0.1844 g TR
0.0482 g B3 amide+0.09 g B7 dissolved 0.025 g TR*
0.03175 g B3 amide+0.069 g B1+0.006352 g B7 dissolved 0.025 g TR* *there was immediately precipitate.

Testing was done with flavored glycerin (with 2,3 butane diol added) instead of the regular stock and this had lower solubility of TR than the baseline (0.045 g/ml). Currently piperazine hexahydrate is being tested as it is another hydrotrope. 0.000263 mol of piperazine.6H₂O in 1 ml of stock had 0.0731 g of TR added to it and after a few days the solution became dark and there was a small amount of precipitate. It may work better with other co-solutes or ethanol.

Further experiments would study the effectiveness of other hydrotropes, as niacinamide is generally the weakest at increasing solubility (see attached papers). These other hydrotropes include but are not limited to: catechol, pyrogallol, resorcinol, isosorbide. Combinations of them are supposedly more effective.

Piracetam. This is a well-studied nootropic (cognitive enhancer). It is very polar and water-soluble. Its solubility in stock was found to be 36.5 mg/ml. Its solubility may be enhanced by adding water to the solution, using very polar cosolvents (propylene carbonate, ethylene carbonate) and adjusting the ion concentration in the solution (salting in/out). However it's effective dosage is at least 1.6 g/day for healthy individuals and a more potent racetam would be a better choice. These include but are not limited to aniracetam, oxiracetam, phenylpiracetam, couracetam, and pramiracetam. These other compounds are generally hydrophobic and have very unpleasant taste. Their much higher potency (2-3 orders of magnitude) and low oral bioavailability compared to piracetam and are much better candidates for vaping.

Pantothenic Acid (Vitamin B5). This vitamin helps provide energy for your body and is found in many energy drinks and supplements. Currently it is being tested in the pure stock and has a solubility of at least 30 mg/ml when added incrementally and heated.

### Example 2

The following provides an example of administration of a composition of the present disclosure.

The term “vape” is used herein as a verb to describe the action of utilizing a device to inhale the composition. The subject was under fasting conditions for greater than 12 hours prior to vaping. Approximately 10 ml of blood was drawn before the assay as a baseline using a “red-top” tube, viz., an evacuated Beckton-Dickinson blood-draw tube with no additives or anticoagulants. All blood-draw tubes were of the so-called “red top” type unless otherwise indicated. This entire experiment was performed with an observer present at all times who recorded the data.

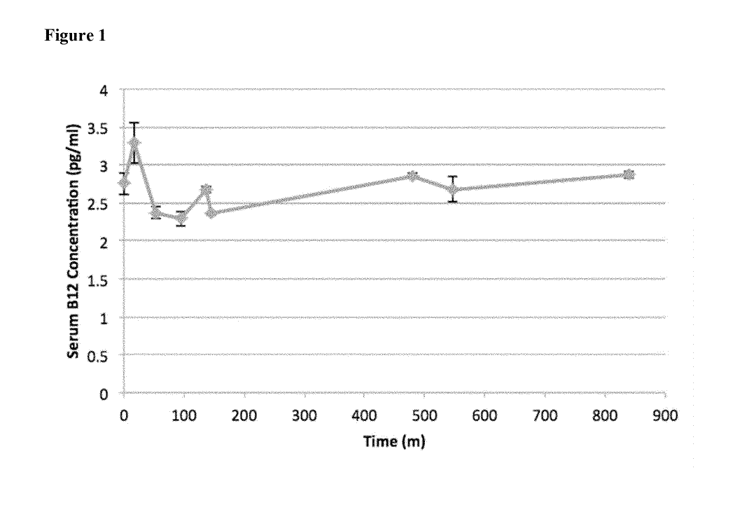

The subject utilized an incentive spirometer to measure the approximate pulmonary inspiration volume, taking care to not hyperinspire beyond typical, resting, lung capacity. The liquid-state concentration of the B12 composition was 0.21M. The subject then began the experiment at time t=0 with the inhalation volume of approximately 1 L of vapor. This was performed three times before the first blood draw at 18:50 minutes. The subject then continued to vape at reasonable intervals between blood draws and all samples were labelled with times. See FIG. 1.

The collected blood specimens were then spun down in an IEC-brand centrifuge at approximately 400 Hz for 0.5 hour. Serum from each tube was then collected using standard laboratory techniques and 200 ul aliquots were pipetted into microcentrifuge tubes, labelled and capped. An Accubind® B12 ELISA kit (Monobind, LLC: model 7625-300B) was used and instructions were followed as provided by the manufacturer kit. The microplate containing the duplicated samples with controls was then read by a Biotek Microplate Reader using a 450 nm filter, per the ELISA product manufacturer instructions.

### Example 3

The following provides examples of preparation of compositions of the present disclosure.

### Experimental Procedures

Folate: To a test tube of 3.5 ml of (0.5 mol of glycerin and 0.5 mol of propylene glycol), 0.5 ml of water, 0.5 ml of ethanol, 0.125 ml of flavored vodka, and 0.1 ml of flavored glycerin 8 mg of folic acid, 22.1 mg of nicotinamide, and 4.7 mg of biotin were added incrementally and heated at 175° C. each time; resulting in a clear solution with flavor.

Vitamin D3: To a test tube of 2.5 ml of (0.7 mol of glycerin and 0.3 mol of 2,3 butane diol) and 1.0 ml of water, 5 mg of Vitamin D3, 15 mg of βcyclodextrin, 15 mg of cinnamic acid, and 15 mg of ethyl vanillin were added incrementally and heated at 175° C. each time; resulting in a clear solution with flavor.

Piracetam: To a test tube of 1.5 ml of (0.7 mol of glycerin and 0.3 mol of 2,3 butane diol), 2.0 ml of fruit flavored vodka, and 0.1 ml of flavored glycerin, 0.2 grams of piracetam were added incrementally and heated at 175° C. each time; resulting in a clear solution with flavor.

Vitamin B5: To a test tube of 1.5 ml of (0.7 mol of glycerin and 0.3 mol of 2,3 butane diol), 2.0 ml of fruit flavored vodka, and 0.1 ml of flavored glycerin, 0.2 g of vitamin B5 were added; resulting in a clear and flavored solution.

L-Theanine/L-Arginine: To a test tube of 0.75 ml of (0.7 mol of glycerin and 0.3 mol of 2,3 butane diol), 2.5 ml of water, and 0.25 ml of flavored glycerin, 0.693 g of L-arginine and 0.693 g of l-theanine were added; resulting in a clear and flavored solution.

Vitamin B2: To a test tube of 1.5 ml of (0.7 mol of glycerin and 0.3 mol of 2,3 butane diol), and 2.0 ml of 0.1 mol of Na₂CO₃, 9.26 mg of 1-arginine and 18 mg of riboflavin were added resulting in a clear solution.

Noopept: To a test tube of 2.0 ml of (0.7 mol of glycerin and 0.3 mol of 2,3 butane diol), 2.0 ml of ethanol, and 0.1 ml of flavored glycerin, 0.335 g of noopept and 58 mg of cinnamic acid were added; resulting in a clear and flavored solution.

Trans Resveratrol: To a test tube of 3.5 ml of (0.7 mol of glycerin and 0.3 mol of 2,3 butane diol), 0.2 g of trans resveratrol, 0.12 g of nicotinamide, 0.08 g of ethyl vanillin, 0.06 g of cinnamic acid; resulting in a clear and flavored solution that became unstable after two weeks and precipitated.

B12: 850 mg of B12 was initially added to 25 ml water. Then, 15 ml of 70% glycerin/30% 2,3 butane diol (v/v) was added and the mixture stirred under mild heat (35° C.). Upon dissolution, 2.5 ml of 0.01M NaOH was added, then another 2.68 g of B12 was added and the contents stirred until dissolution. 13.46 ml of 95% EtOH was then added to the mixture under the same heating/stirring conditions until the solution went from turbid to translucent. To this now dissolved system, another 2.5 ml of 0.01M NaOH, followed by another 8.12 g of B12 was added. The solution was raised to 40° C. and allowed to stir for 30 minutes until the solution again became translucent. Another 2.5 ml of 0.01M NaOH was added and the final 4.26 g of B12 was added. The system was allowed to cool to room temperature, with stirring, overnight. The final, translucent solution was ˜0.21M.

Although the present disclosure has been described with respect to one or more particular embodiments and/or examples, it will be understood that other embodiments and/or examples of the present disclosure may be made without departing from the scope of the present disclosure.

What is claimed is:

1. A composition for vapor phase delivery of active compounds comprising:
a) one or more polyol;
b) optionally, one or more disruptive compound; and
c) one or more active compound.

2. The composition of claim 1, wherein the composition further comprises a solubilizing compound.

3. The composition of claim 1, wherein the composition further comprises an additional compound.

4. The composition of claim 1, wherein the composition further comprises an organic solvent and/or water.

5. The composition of claim 1, wherein the polyol is selected from the group consisting of diols, triols, tetraols, pentaols, hexols, heptols, polyol ethers, and combinations thereof.

6. The composition of claim 5, wherein the polyol is selected from the group consisting of 1,3-butanediol, 1,2-butanediol, 2,3-butanediol, 1,2,5-pentanetriol, 1,3,5-pentanetriol, 2,4-pentanediol, 1,2-pentanediol, 1,2,6-hexanetriol, 1,2,3-propanetriol, (2R, 3S) butane-1,2,3,4-tetraol, (2R,3R)-butane-1,2,3,4-tetraol, (2R,4R)-pentane-1,2,3,4,5-pentol. (2R,4S)-pentane-1,2,3,4,5-pentol, (2R,3S,4S)-pentane-1,2,3,4,5-pentol, and (2R,3S,4R,5S)-hexane-1,2,3,4,5-pentol, (2R,3R,4R,5R)-hexan-1,2,3,4,5,6-hexol, (2S,3R,4R,5R)-hexane-1,2,3,4,5,6-hexol, (2R,3S,4R,5S)-hexane-1,2,3,4,5,6-hexol, and (2R,3S,4S,5R)-hexane-1,2,3,4,5,6-hexol, (2R,3R,5R,6R)-heptane-1,2,3,4,5,6,7-heptol, (2S,3S,5S,6S)-heptane-1,2,3,4,5,6,7-heptol, ((2R,3R,4R,5R)-6-[[(2S,3R,4S,5S,6R)-3,4,5-trihydroxy-6-(hydroxymethyl)-2-tetrahydropyranyl]oxy]hexane-1,2,3,4,5-pentol), (4-O-α-D-galactopyranosyl-D-glucitol), (4-O-α-D-glucopyranosyl-D-glucitol), and combinations thereof.

7. The composition of claim 1, wherein the polyol is racemic 2,3 butane diol and/or 1,2,3-propanetriol.

8. The composition of claim 1, wherein the disrupting compound is selected from the group consisting of bases, C₁ to C₆ linear aliphatic alcohols with a single hydroxyl group, linear carbohydrates, chelating compounds, cage molecules, hydrotropes, chaotropes, kosmotropes, surfactants, and combinations thereof.

9. The composition of claim 1, wherein the disrupting compound is ethanol.

10. The composition of claim 1, wherein the active compound is selected from the group consisting of vitamins and their vitamers, and derivatives thereof, antioxidants, hormones, diarylheptanoids, zwitterionic dipeptides, flavonoids, phenylethanoids, polyphenols, benzopyrones, naturally occurring phenols, flavanols, procyanidins, carotenoids, xanthophils, phenylpropanoids, glycosides, amino acids and their derivatives, phospholipids; omega-3-fatty acids; racetams; peptides; purines, nitrogenous bases, alkaloids, alkaloid derivatives, immunodilators, pure, freebase crystals derived from plants, plant extracts; carboxylic acids, ACE inhibitors, synthetic hydroxymethylphenols, sulfonamides, amides, methyl-n-oates, heptanoic acids, sulfonamides, amides, conjugate bases, drugs, and combinations thereof.

11. The composition of claim 10, wherein the active compound is selected from the group consisting of retinol, beta-carotene, alpha-carotene, gamma-carotene, beta-cryptoxanthin, thiamine, riboflavin, niacin, niacinamide, pantothenic acid, pyridoxine, biotin, folate, cyanocobalamin, ascorbic acid, vitamin D1, vitamin D2, vitamin D3, vitamin D4, vitamin D5, α-tocopherol, tocotrienol, γ-tocopherol, vitamin K1, vitamin K2, trans resveratrol, n-acetylcysteine, melatonin, insulin, insulin hexamer, estradiol, testosterone, oxytosin, curcumin and demethoxycurcumin, carnosine, acetyl carnosine, flavan-3-ol, catechin, epicatechin gallate, myricetin, gallocatechol, tyrosol, hydroxytyrosol, acetyl hydroxytyrosol, kaempferol, galangin, carbenoxolone, ferulic acid, caffeic acid phenethyl ester, fraxetin, echinacea alkamides, Co Q10, tyrosol, and curcumin, α-δ carotene, β-cryptoxanthin, neoxanthin, echinacoside, caffeic acid glycoside, carnitine, acetyl carnitine, choline, cytidine phosphate choline, CDP choline, alpha GPC, phosphatidylcholine, tyrosine and acetyl 1-tyrosine, modafinil, adrafinil, armodafinil, phosphatidylserine, phosphatidylcholine, DHA, EPA, alpha linolenic acid, piracetam, aniracetam, oxiracetam, phenylpiracetam, fasoracetam, coluracetam, pramiracetam, tianeptine, noöpept, selank, acetyl selank, semax, acetyl semax, and fabomotizole, 5-HTP, l-theanine, caffeine, phenibut; picamilon; pyritinol; sulbutiamine, huperzine A, vinpocetine, vincamine, uridine monophosphate, zinc citrate, β-glucan, arginine, l-theanine; Lion's Mane Mushroom, Rhodiola Rosea, Cordyceps, Garlic, Astralagus, St. John's Wort, Milk Thistle, Ginger, Ginseng, Echinacea, Eleuthero, Ashwagandha, Dangshen, albuterol, diuril, nialamide, furosemide, torsemide, lovastatin, atorvastatin, amlodipine, selegiline, guaifenesin, tyloxapol, acetylcysteine, dextromethorphan, diphenhydramine, loratidine, phenylephrine HCl, theophylline, berberine, piperine, chelerythrine, quinidine, synribo, ephedrine, fucoxanthin, fenugreek, hordenine, sildenafil, mannitol, xylitol, trigonelline, and combinations thereof.

12. A method for vapor phase delivery of an active compound or compounds comprising:
administering an active compound or active compounds in the vapor phase to an individual using a composition of claim 1.

13. The method of claim 12, wherein after administration of the compound the individual has a detectible amount of the active compound(s) or a derivative of the active compound(s) (e.g., one or more metabolite(s)) in its blood and/or urine.

14. The method of claim 12, wherein the active compound is rendered into the vapor phase using a vaporizing apparatus.

15. The method of claim 14, wherein the vaporizing apparatus is an electronic vaporizing apparatus.

16. A kit comprising:
a) one or more compositions of claim 1; and
b) instructions for use of the one or more compositions.

17. The kit of claim 16, further comprising a vaporizing apparatus.

18. The kit of claim 17, wherein the vaporizing apparatus is an electronic apparatus.

19. The kit of claim 16, wherein the instructions describe how to administer the one or more compositions to an individual.
